(19) United States
(12) Patent Application Publication
(10) Pub. No.: US 2009/0306643 A1
(43) Pub. Date: Dec. 10, 2009

(54) METHOD AND APPARATUS FOR DELIVERY AND DETECTION OF TRANSMURAL CARDIAC ABLATION LESIONS

(76) Inventors: Carlo Pappone, Milano (IT); Nathan Kastelein, St. Louis, MO (US)

Correspondence Address: HARNESS, DICKEY, & PIERCE, P.L.C, 7700 Bonhomme, Suite 400, ST. LOUIS, MO, 63105, US

(21) Appl. No.: 12/392,497

(22) Filed: Feb. 25, 2009

Related U.S. Application Data
(60) Provisional application No. 61/031,318, filed on Feb. 25, 2008.

(51) Int. Cl. A61B 18/18

(52) U.S. Cl. 606/33

(57) ABSTRACT

During cardiac wall tissue ablation with an RF catheter, the observation of an 8 to 12 ohm drop in the tissue impedance is indicative of the production of a transmural lesion. Due to a magnetic catheter's ability to stay in the same position throughout the cardiac cycle and the consistency of forces applied throughout the cardiac cycle, the impedance measurement from the distal electrode of the magnetic catheter is uniquely useful in determining the achievement of a transmural lesion. The use of this impedance measurement during ablation with a magnetic catheter can thus be used as an indication of when the ablation has achieved a successful treatment endpoint. An RF generator's impedance measurement along with knowledge of the navigational state of a magnetic catheter can thus be used to control the delivery of energy for the purpose of delivering only as much RF energy as is necessary to achieve a clinically effective lesion and to stop RF energy delivery prior to the onset of an adverse event.

### CROSS-REFERENCE TO RELATED APPLICATIONS

This application claims priority to U.S. Provisional Patent Application Ser. No. 61/031,318, filed Feb. 25, 2008. The disclosure of the above-referenced application is incorporated herein by reference.

### FIELD

The present disclosure relates to navigation of medical devices within a subjects body, including complex composite surgical devices, and more particularly to the use of magnetic navigation for the performance of heart surgery interventions, such as electrophysiology ablation therapy.

### BACKGROUND

A variety of techniques are currently available to physicians for performing minimally invasive cardiac electrical and electrophysiological disorder repair. For example, magnetic steering techniques provide computer-assisted control of a catheter tip while allowing an operating physician to remain outside the operating room x-ray field.

When navigating medical devices by mechanical means, the need to transfer a proximally applied push force, and more critically, the need to effect a distal rotation through proximally applied torque leads to a relatively high device stiffness requirement. Device stiffness, in turn, limits device tip flexibility, maneuverability, and ability to maintain tissue contact during a cardiac cycle, resulting in relatively unpredictable ablation properties and therapy results.

### SUMMARY

The present invention relates to the navigation of medical devices for surgical heart interventions, such as heart wall tissue ablation and cardiac rhythm restoration in electrophysiology procedures, and similar minimally invasive heart surgeries.

In one embodiment of the present invention, medical devices enabling improved ablation therapy control and performance are disclosed.

In another embodiment of the present invention, various embodiments of a method are disclosed that facilitate control of an ablation therapy by providing well-defined, measurable, and unambiguous local ablation endpoint measures.

In a further aspect of the present invention, various embodiments of a system for the improved performance of ablative heart therapy and related procedures are disclosed.

Further areas of applicability will become apparent from the description provided herein. It should be understood that the description and specific examples are intended for purposes of illustration only and are not intended to limit the scope of the present disclosure.

### BRIEF DESCRIPTION

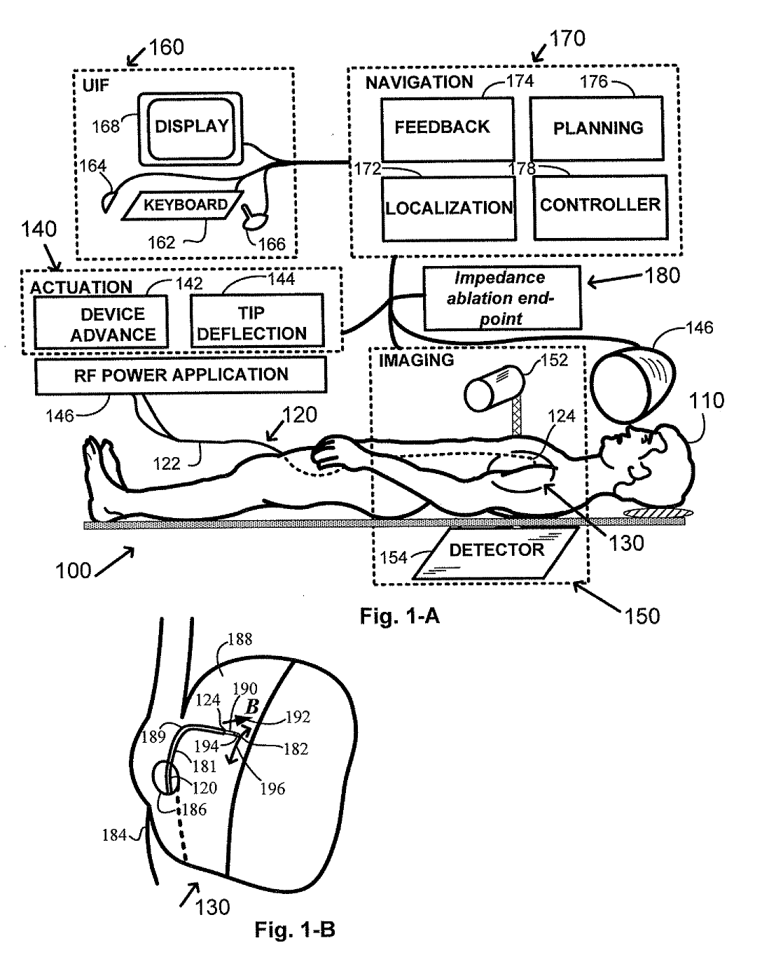

FIG. 1-A is a system block-diagram of one embodiment of a magnetic navigation system for minimally invasive electrophysiological heart surgery and related interventions;

FIG. 1-B is a schematic illustration of a heart, showing a medical device that has been navigated to the right atrium of the heart and being used to perform an atrial wall tissue ablation;

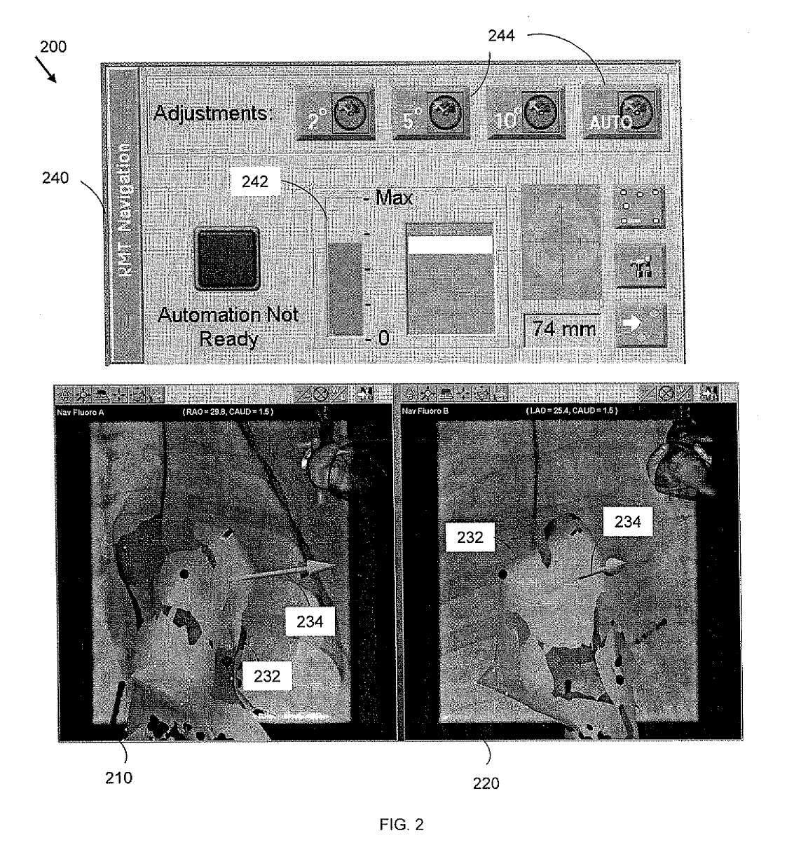

FIG. 2 is a view of one possible embodiment of a contact meter user interface display and user interfaces for the monitoring and controlling therapeutic localized tissue ablation;

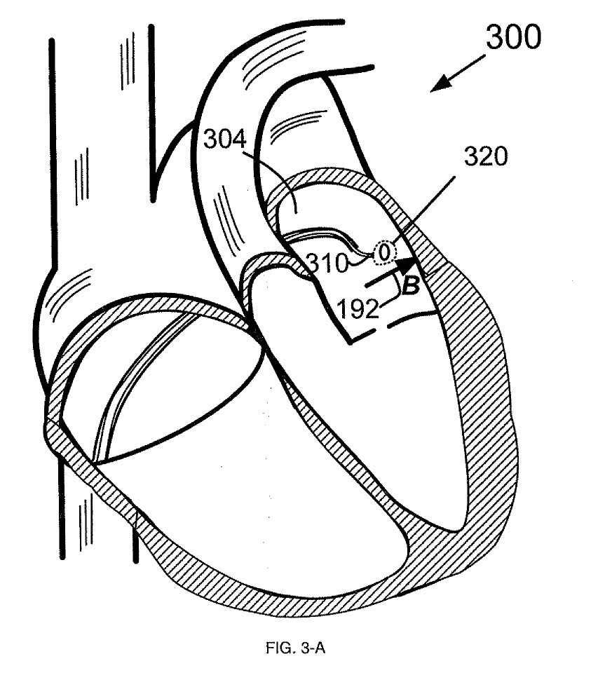

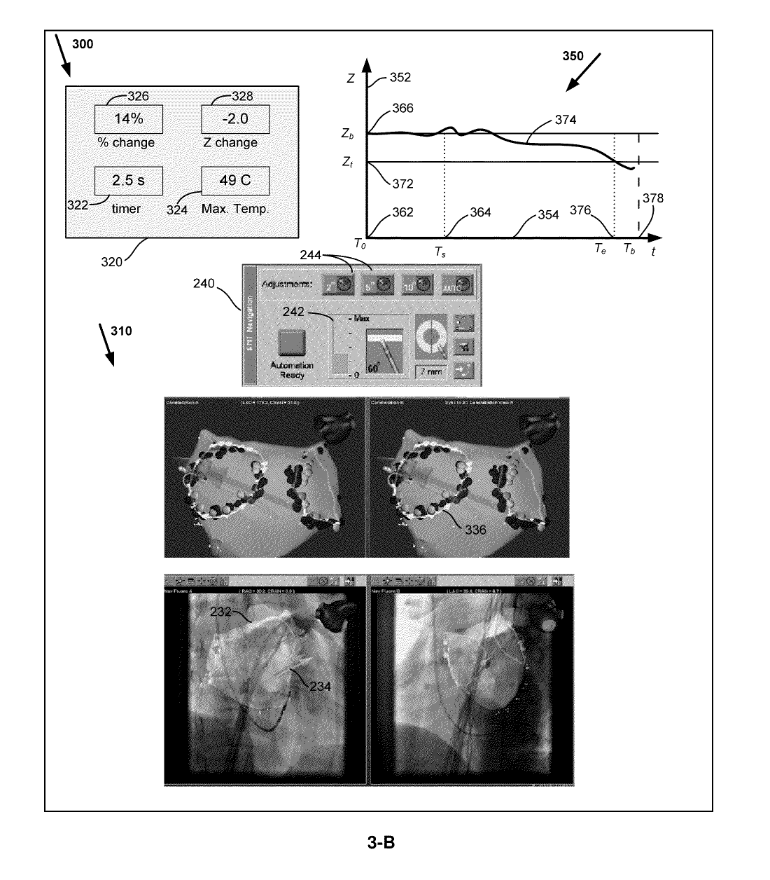

FIG. 3-A is a schematic illustration of a heart, showing cardiac tissue ablation around a pulmonary vein in the left atrium of the heart;

FIG. 3-B is a view of the displays of one possible embodiment of a user interface during cardiac tissue ablation around a pulmonary vein in the left atrium of the heart, and a user interface panel displaying a calibrated tissue impedance time graph and associated control parameters; and

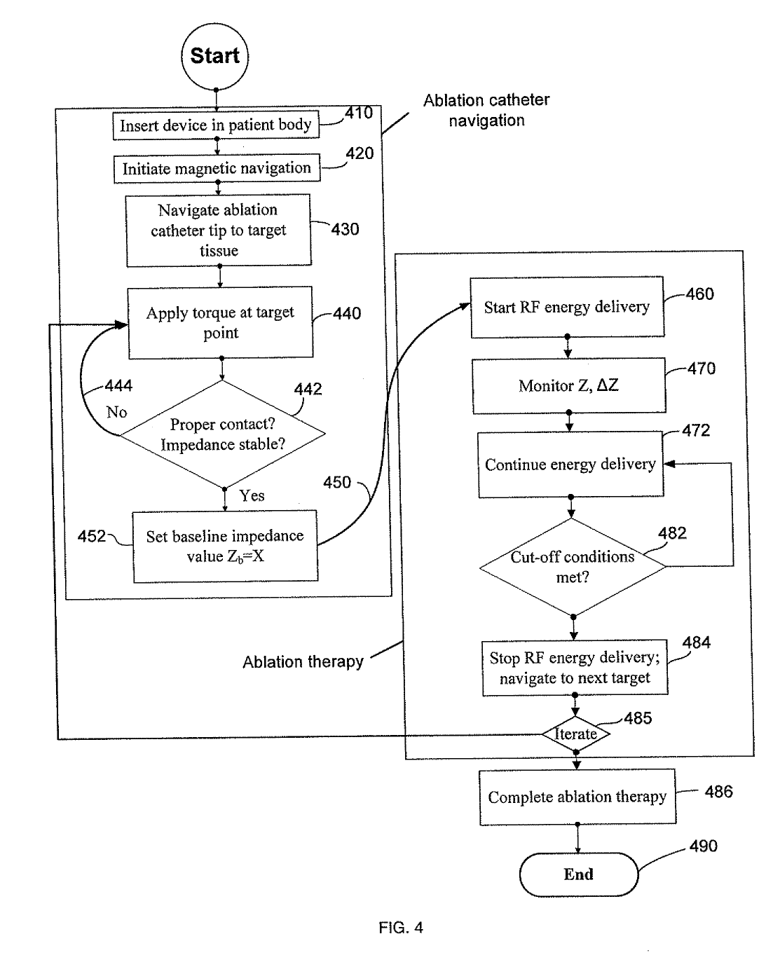

FIG. 4 is a flow chart of one embodiment of the present invention as applied to the determination of an ablation therapy endpoint for a given location on a selected ablation path on a heart wall surface.

Throughout the drawings, corresponding reference numerals indicate like or corresponding parts and features.

### DETAILED DESCRIPTION OF THE INVENTION

The various embodiments of the invention provide for devices, methods, and systems for enhanced performance of ablative procedures within a subject's body through the use of specifically designed measurement instruments, controls, ablative energy devices, guidewires, and catheters. These improvements can lead to highly accurate device positioning, significantly shorter intervention times, and improved cardiac therapy results.

An elongate navigable medical device 120 having a proximal end 122 and a distal end or tip 124 is provided for use in an interventional system 100, as shown in FIG. 1-A. A subject 110 is positioned within the interventional system, and the medical device 120 is inserted into a blood vessel of the patient and navigated to an intervention volume 130. In magnetic navigation, a magnetic field externally generated by magnet(s) 146 orients a small magnet located at the device distal end (not shown). Real-time information is provided to the physician for example, by an x-ray imaging chain 150 comprising an x-ray tube 152 and an x-ray detector 154, and also possibly by use of a three-dimensional device localization system, such as a set of electromagnetic receivers located at the device distal end (not shown) and associated external electromagnetic emitters, or other localization devices with similar effect. The physician provides inputs to the navigation system through a navigation computer 160 comprising user interface devices, such as a display 168, a keyboard 162, mouse 164, joystick 166, and similar input devices. Display 168 also shows real-time image information acquired by the imaging chain 150 and surface rendering information generated from data acquired by the three-dimensional localization system. Computer 160 relays inputs from the user to a controller 178 that determines and effects the magnet(s) orientation through actuation control 140.

As shown in FIG. 1-B, the medical device 120 has been navigated successively to and through the right atrium of the heart. In specific embodiments, device tip(s) 124 also has sensor(s) (not shown), such as strain gauges or similar devices located at or near the distal end to provide force data information to estimate the amount of pressure applied on the target tissue 182, and/or as feedback to navigation sub-system 170 in assisting navigation. Other sensors might include an ultrasound device or other device appropriate for the determination of distance from the device tip to the tissue. Additional force sensors may be provided along various device segments to measure the amount of force exerted by the subject's tissues onto the device. Such sensors signals, including feedback data from the device, are processed by feedback block 174, which in turn communicates with control block 178, as well as with UIF sub-system 160. Further device tip feedback data include relative tip and tissue positional information provided by an imaging system or a device localization system, and predictive device modeling. In particular, feedback information is processed to generate a device tip contact quality measure that enables improved ablation therapy performance by indicating whether contact quality is sufficient for application of ablative energy. In some embodiments if contact quality is inadequate, the system may suggest corrective actions.

In closed loop implementations, navigation controller 178 automatically provides input commands to the system magnet(s) and device actuation sub-system 140, based on feedback data and previously provided navigation input instructions. In semi-closed loop implementations, the physician fine-tunes the navigation control, based in part upon feedback and imaging data. Control commands and feedback data may be communicated from the user interface 160 and controller 178 to the device and from the device, back to the feedback block 174, through cables or other means, such as wireless communications and interfaces.

As known in the art, system 100 comprises an electromechanical device actuation block 140 controlling a device advancer 142 capable of precise device advance and retraction, based on corresponding control commands. Deflection actuation sub-block 144, controls device tip deflection; several deflection modalities that allow computer controlled navigation are known in the art, such as magnetic navigation, mechanical pull wire actuation, electrostrictive or magnetostrictive deflection, hydraulic methods, among others. In specific applications, such as in electrophysiology, cardiac wall tissue ablation is performed in order to destroy diseased tissues, including sites of spurious secondary electrical activity or to isolate such sites from essential cardiac structures that may otherwise suffer from fibrillation or asynchronous stimulation. Block 180 in FIG. 1-A, represents schematically the use of a specific impedance measure as an ablation end-point.

FIG. 1-B further shows a composite medical device 120 comprising a sheath 181, and an ablation catheter 190, the composite device having progressed through the lower vena cava 184, and through the vena cava ostium 186, into the right atrium of the heart 188. The sheath may comprise a J-shaped bend 189 near its distal end to provide additional catheter support. The ablation catheter 190, is guided therethrough to the sheath distal end 124, and beyond, through application of a variable navigation magnetic field 192. The ablation catheter comprises a distal tip magnet 194 and an ablation electrode (not shown in the figure).

After having been navigated to contact the atrial wall at precise target location 182, and subsequent to verification of the quality of contact between the catheter tip and the target tissues, the ablation catheter electrode is energized to perform cardiac wall tissue ablation per the therapeutic needs established during electrophysiological disorder diagnostic and characterization. During the ablation time, the tissue target 182 moves as a consequence of the cardiac rhythm, as schematically illustrated by arrow 196 The specific magnetic navigation catheter features, including softness and flexibility, make it possible for the ablation catheter tip to remain precisely positioned on the cardiac wall at tissue target 182 during the entire ablation treatment. Further, these features also allow maintaining a device tip contact quality appropriate for ablation energy delivery during one or more cardiac cycles within the ablative phase.

Radio-frequency (RF) power delivery and resulting tissue ablation treatment, constitutes a standard interventional procedure part of the arsenal of therapies available to modern medicine for the treatment of cardiac arrhythmias. It is usually performed with a catheter comprising a shaped conducting electrode tip that when energized, delivers RF energy to cardiac tissue. While such procedures are often performed manually, technological advances have been implemented that enable computer-controlled navigational steering and improved access to desired cardiac target locations. Such novel technologies include magnetic navigation systems and advances made thereto.

An example of such advances relating to device distal orientation and associated contact quality control is described in PCT application PCT/US05/46641, incorporated herein by reference. In PCT application PCT/US05/46641 assigned to Stereotaxis and entitled “contact over torque with three-dimensional anatomical data,” a method of improving contact between a magnetic catheter distal tip and a three-dimensional tissue surface is disclosed that comprises obtaining a target location on the surface for the device tip to contact, obtaining local surface geometry information in a neighborhood of the target location, and using this information to determine a change of at least one control variable for effecting an over-torque of the medical device to enhance contact of the device with the target surface.

FIG. 2 presents schematically, as generally indicated by numeral 200, one embodiment of a user interface display and controls for tip contact quality, as known in the prior art. In FIG. 2 the displays present two x-ray projections, one in the right-anterior oblique (RAO) view 210, and one in the left-anterior oblique (LAO) 220. Both views also include a cranio-caudal angulation component. On both of these views, a heart surface rendering 232 is superimposed; the data for the surface generation having been collected through a sampling of the heart volume using a localization device. Also shown on both views is a current direction of the applied magnetic field 234. FIG. 2 further shows a user interface panel 240 indicating, among other parameters, the current degree of contact quality 242, as well as a number of adjustment changes 244 that the user can prescribe to improve contact quality.

In general, when a lesion is created by delivery of RF energy, achieving a transmural lesion is a desirable feature, where the lesion extends most or all the way through the cardiac wall thickness. However, for safety reasons, the lesion must not create a hole or perforation through the cardiac wall. In current practice, as RF energy is being delivered, the temperature at the tip of the catheter is monitored with the aid of a thermocouple, or other temperature sensing device embedded in the catheter tip, and a temperature cutoff limits RF energy delivery to prevent excessive ablation. Unfortunately in some cases, the catheter tip temperature is highly dependent on unknown local conditions near the catheter tip, and the local tissue temperature of relevance can in fact, be quite different from the measured catheter tip temperature.

Therefore, there is a need to provide a reliable parametric measure of when RF energy delivery should cease during ablative therapy. The present invention provides such a measure whenever a stable catheter tip-tissue contact exists, as is typically the case when using a magnetic navigation system to steer an interventional device distal tip and maintain its contact with tissue as well as to maintain contact quality.

In a magnetic navigation system, external magnets are used to generate a desired magnetic field 192 within the navigation volume, as illustrated in FIG. 3-A, whereby a magnet-tipped interventional device 310 may be steered within the anatomy in a finely controlled manner. Catheters designed for use with a magnetic navigation system tend to be flexible and soft in the distal portion, and as a result, remain highly navigable even with the full length of the device inserted into and engaged with narrow lumen anatomy. An additional benefit provided by these catheters is that the distal tip of the device tends to maintain contact with the cardiac wall during wall motion through the cardiac cycle. The applied magnetic field bends the distal portion of the device and works to align it with the magnetic field.

If the tip of the device is contacting a given location on the cardiac wall, as the wall moves with the heartbeat, the tip will tend to maintain contact at the same wall location due to a combination of two factors: the tendency of the tip to stay aligned with the magnetic field and the flexible nature of the catheter shaft that allows it to easily buckle proximally to the tip magnet as the wall moves. Furthermore, the variation in tip/tissue contact force over a cardiac cycle is smaller for a (soft) magnetic catheter than it is for stiffer non-magnetic catheters, resulting in generally more consistent contact force and overall contact quality over a cardiac cycle. These properties lead to increased stability in tip/tissue impedance readings, enabling the use of contact impedance as a parameter that can be monitored to indicate sufficient delivery of RF energy for transmural ablation. The magnetic catheter is also equipped with a sensor for obtaining high-resolution position and orientation information associated with the catheter tip. This information can be used by the magnetic navigation system to enhance contact and ensure that stable and high-quality tip/tissue contact is maintained.

In the application, schematically illustrated in FIG. 3-A, an ablation catheter traces a series of points around a pulmonary vein ostium in the left atrium 304, thereby electrically insulating the heart chamber from spurious electrical signals arising at the vein ostium or within the vein itself. At each point on the ablation path 320, the ablation distal tip is positioned in stable contact with the cardiac tissue, and RF energy delivery proceeds until an appropriate ablation end-point has been reached.

A key observation underlying the present invention is that during ablation with a magnetic catheter, a drop in local impedance (as measured through the catheter tip) occurs, with the drop from baseline (pre-ablative) to post-ablative impedance in the 5-12 ohm range. More specifically, a drop in measured impedance value of magnitude in the range 8-12 ohm typically indicates that sufficient RF energy has been delivered to obtain a transmural lesion. This drop in impedance value of a magnetic catheter in stable contact with the cardiac wall can thus be used as an indication of transmural lesion achievement, and delivery of RF energy can be stopped when this drop has been measured or observed, Alternatively to an absolute value of the impedance drop, a percentage drop in impedance can also be used as a measure to specify sufficiency of RF energy delivery; thus, starting from a baseline impedance value at the target location, an impedance drop in the approximate range 5-20%, and more specifically in the range 8-15%, indicates creation of a transmural lesion.

FIG. 3-B presents a panel from a user interface and images from an actual intra-cardiac tissue ablation procedure around a pulmonary vein in the left atrium of the heart. The figure also illustrates the displays and interface 310 discussed in relation to FIG. 2, including the three-dimensional localization map rendition 232, target magnetic field vector 234, user interface panel 240, including contact quality indicator 242, and adjustments to be selected 244 to improve contact quality. Additionally, the figure also presents one embodiment of a user interface panel 320 for ablation control at a given point. This interface comprises a back-up time 322, setting the maximum time for application of ablative energy at a single point; a maximum tissue temperature 324, beyond which ablative energy application ceases; and two calibrated impedance change thresholds 326 and 328, that respectively allow input of the target percentage change and an absolute impedance change, either of which (or both), being selectable as indicative of ablation performance completion, and as a result, obtainment of a transmural lesion. FIG. 3-B also presents an impedance graph 350, showing a plot of impedance Z 352 as a function of elapsed time t 354. The impedance graph shows a period between a time Tₒ 362 and a time Tₛ 364 during which calibration and base-line impedance data are acquired, leading to the definition of an impedance base line Z_{b} 366, a target impedance value Zₜ 372 indicative of ablation completion, and a backup time T_{b} 378. At time Tₛ 364 the application of ablative energy starts and continues till either the impedance data line 374 crosses the target impedance value, as indicated in the graph at point Tₑ 376, or the energy application time reaches its maximum value T_{b} 378. Of course in different implementations, other parameters may be measured and can lead to cessation of ablative energy application; such parameters include a local tissue temperature, ECG extracted parameter values and other such relevant parameters, as known in the art.

As illustrated in FIG. 4, an embodiment of a method or workflow for the delivery of ablation and detection of transmural intra-cardiac ablation lesions proceeds as follows:
1. Upon procedure start, insert the medical device in the patient 410, initiate magnetic navigation 420, and navigate the catheter tip to the desired target location 430; confirm catheter tip placement, for example from intra-cardiac ECG and fluoroscopy information, and apply torque 440 as necessary to properly orient the device distal tip with respect to the tissue wall;
2. Check contact quality 442 from the “Contact Meter” associated with the magnetic navigation system The meter reading is typically a function of the orientational difference between catheter tip orientation and applied magnetic field orientation, and provides a good measure of catheter tip/tissue contact; iterate as necessary;
3. If contact is insufficient, branch 444, apply more magnetic torque at step 440 (using tools available on the navigation system user interface for such control prescriptions) to enhance contact. Correspondingly, the contact meter should indicate enhanced contact;
4. Check that the impedance reading is stable, with a fluctuation range of no more than about plus/minus 2 ohms. Let this baseline value be X, 452;
5. Start RF power delivery 460, with a time or temperature back-up cutoff (as currently practiced);
6. Monitor the impedance Z value 470 during RF power delivery 472, and the change ΔZ in impedance. If cutoff conditions for ΔZ are attained before the time/temperature backup/threshold are reached, stop RF power delivery. Cutoff conditions could be any of: (i) ΔZ reaches or exceeds a pre-determined threshold value (such as for example 10 ohms), or (ii) ΔZ reaches or exceeds a pre-determined fraction (such as for example 0.1) or combinations thereof.
7. Move to the next target 484, and iterate 485, until all ablation targets have been treated, the ablation therapy is complete, 486, and the method ends 490.

It is important to note that the impedance-based ablation cutoff measure could be used by itself in one preferred embodiment, or in an alternative embodiment, it could be combined with an intra-cardiac ECG amplitude-based cutoff. Thus for instance in the latter embodiment, if the intra-cardiac ECG amplitude has dropped by 80%, and the impedance drop has reached a threshold value, then RF energy delivery is stopped.

Such an impedance-based measure of ablation effectiveness is also useful with a high-power RF catheter, such as for instance an irrigated catheter (that uses a flowing saline solution to carry away excess heat from the catheter tip), or with a catheter with a relatively short tip electrode (in the approximate length range 2-4 mm), or both.

In one preferred embodiment, the remote navigation system can communicate with the RF generator and receive realtime data including impedance information, and instruct the RF generator to turn off power delivery when an appropriate impedance endpoint, an ECG-based endpoint, or combination thereof has been achieved.

Thus, an RF generator circuit impedance measurement along with knowledge of the navigational state of a catheter can be used to control the delivery of energy for the purpose of delivering only as much RF energy as is necessary to achieve a clinically effective lesion and to stop RF energy delivery prior to the onset of an adverse event.

Although the present invention has been described with respect to several exemplary embodiments, there are many other variations of the above-described embodiments that will be apparent to those skilled in the art, even where elements have not explicitly been designated as exemplary. It is understood that these modifications are within the teaching of the present invention, which is to be limited only by the claims appended hereto.

What is claimed is:

1. A method of controlling an RF cardiac wall ablation therapy comprising navigating an ablation catheter to a target point, establishing a contact between an ablation catheter distal end and the target point, controlling the contact quality, applying RF energy, monitoring a circuit impedance measure, and stopping RF energy application based at least in part on circuit impedance measurements.

2. The method of claim 1, further comprising establishing a base line impedance value and stopping RF energy application upon a predetermined change in the circuit impedance measure.

3. The method of claim 2, wherein the predetermined change in the circuit impedance measure is a percentage change.

4. The method of claim 2, wherein the predetermined change in the circuit impedance measure is an absolute change.

5. The method of claim 1, further comprising stopping RF energy application after a predetermined elapsed time if the application has not been stopped because of the circuit impedance measurements.

6. The method of claim 1, further comprising stopping RF energy application after a predetermined tissue temperature is reached if the application has not been stopped because of the circuit impedance measurements.

7. The method of claim 1, wherein the circuit impedance measure is calibrated so that changes in circuit impedance are associated to changes in tissue impedance.

8. The method of claim 1, further comprising controlling ablation energy application based on parameters extracted from an ECG data series.

9. The method of claim 1, wherein the step of navigating an ablation catheter comprises at least one of mechanical pull-wire navigation, electrostrictive navigation, hydraulic navigation, magnetostrictive navigation, and magnetic navigation.

10. A minimally invasive interventional navigation system for controlled RF heart tissue ablation comprising: an RF enabled medical device; an RF energy application controller for controlling the application to the RF enabled medial device in response a set change in at least one circuit impedance parameter; and a user interface for setting the change in the at least one circuit impedance parameter.

11. The system of claim 10, further comprising a tissue temperature measurement device and associated controller to interrupt RF energy application based on a maximum pre-set tissue temperature.

12. The system of claim 10, further comprising a back-up timer and associated controller to interrupt RF energy application based on a maximum pre-set elapsed time.

13. The system of claim 10, further comprising a controller to interrupt RF energy application based on a measured contact quality between an RF enabled medical distal tip and a cardiac tissue.

14. The system of claim 10, further including a computer and computer instructions to analyze a circuit impedance data time-series and a controller to interrupt RF energy application based on parameters extracted from the impedance time-series by a processing algorithm.

15. The system of claim 10, further comprising an EGG interface and EGG data series analysis software for extracting selected parameters from said ECG data series, and controlling ablative energy application based at least in part upon said extracted EGG data series parameters.

16. A device for performing controlled RF heart tissue ablation, the device comprising: a circuit impedance measurement instrument; a processor to determine measured circuit impedance changes; computer memory for storing impedance measurement change thresholds; and a controller to interrupt RF energy delivery based on comparison between calibrated measured impedance changes and change thresholds stored in memory.

17. The device of claim 16, wherein the controller interrupts RF energy delivery based on an absolute circuit impedance change.

18. The device of claim 16, wherein the controller interrupts RF energy delivery based on a relative circuit impedance change.

19. The device of claim 16, further comprising a backup timer memory and control to interrupt RF energy delivery based on a maximum energy delivery time.

20. The device of claim 16, further comprising a tissue temperature measurement instrument and a control to interrupt RF energy delivery based on a maximum tissue temperature.

21. A method of controlling the RF ablation of tissue with an RF ablation instrument, the method comprising stopping RF ablation based upon a predetermined change in a measured parameter corresponding to impedance of the tissue being ablated.

22. The method according to claim 21, wherein the ablation is stopped based upon a predetermined absolute change in the parameter corresponding to impedance of the tissue being ablated.

23. The method according to claim 21, wherein the ablation is stopped based upon a predetermined relative change in the parameter corresponding to impedance of the tissue being ablated.

24. The method according to claim 21, wherein measured parameter corresponding to impedance of the tissue being ablated is the circuit impedance of the RF ablation apparatus.

25. The method according to claim 21, further comprising stopping the RF ablation if a predetermined time elapses before the predetermined change in a measured parameter corresponding to impedance of the tissue being ablated occurs.

26. The method according to claim 21, further comprising stopping the RF ablation if a predetermined tissue temperature is reached before the predetermined change in a measured parameter corresponding to impedance of the tissue being ablated occurs.
